(19) United States
(12) Patent Application Publication
(10) Pub. No.: US 2025/0289134 A1
(43) Pub. Date: Sep. 18, 2025

(54) SAFETY PARAMETER-BASED TOUCH-INTERACTION CONTROL OF HUMAN-MACHINE INTERACTION DEVICE

(71) Applicant: Honda Motor Co., Ltd., Tokyo (JP)

(72) Inventors: YUHAN HU, SANTA CLARA, CA (US); RANA SOLTANI ZARRIN, LOS GATOS, CA (US)

(21) Appl. No.: 18/733,687

(22) Filed: Jun. 4, 2024

Related U.S. Application Data
(60) Provisional application No. 63/564,897, filed on Mar. 13, 2024.

(51) Int. Cl. B25J 9/16; B25J 13/08

(52) U.S. Cl. CPC B25J 9/1674; B25J 13/081

(57) ABSTRACT

An electronic device and a method for safety parameter-based touch-interaction control of a human-machine interaction (HMI) device. The electronic device receives touch parameters and control parameters to control a physical-interaction of the HMI device and the user. The electronic device controls the physical interaction of the HMI device, based on the received touch parameters and the received control parameters. The electronic device determines a physical response of the user, based on the control of the physical interaction of the HMI device. The electronic device may determine safety metrics associated with the user, based on the determined physical response and the received touch parameters. Furthermore, electronic device determines correlation information based on the control of the physical-interaction of the HMI device and the determined safety metrics. The electronic device to controls the physical-interaction of the HMI device, further based on the determined correlation information.

### CROSS-REFERENCE TO RELATED APPLICATIONS/INCORPORATION BY REFERENCE

This Application also makes reference to U.S. Provisional Application Ser. No. 63/564,897, which was filed on Mar. 13, 2024. The above stated Patent Application is hereby incorporated herein by reference in its entirety.

### BACKGROUND

The rapid advancements in engineering and technology have given rise to a diverse array of human-machine interaction (HMI) devices, including robots, which are now integral to executing complex and critical tasks, such as remote surgeries. The implementation of safety and control protocols in robotic systems is needed for protecting individuals from the potential risks associated with robotic operations. Ensuring robotic safety involves minimizing human exposure to rigorous tasks, thus preventing injuries from repetitive or heavy lifting and mitigating worker fatigue. However, the integration of robots also presents new challenges, including ergonomic issues, potential control system errors, and the necessity for comprehensive training. Robotic safety encompasses not only physical protection but also digital security and psychological well-being, necessitating oversight by regulatory entities and industry leaders to maintain safety standards. Current robotic systems may not fully account for the subtleties of physical human-robot interaction (pHRI), especially in teleoperated robots within healthcare environments. Moreover, these systems might not effectively capture the experiences of both an operator and an end-user (such as a patient), which are mediated through the robot's interface. Also, it is essential to address these gaps to enhance the safety and efficacy of robotic applications in sensitive settings like healthcare.

Further limitations and disadvantages of conventional and traditional approaches will become apparent to one of skill in the art, through comparison of described systems with some aspects of the present disclosure, as set forth in the remainder of the present application and with reference to the drawings.

### SUMMARY

According to an embodiment of the disclosure, an electronic device for safety parameter-based touch-interaction control of human-machine interaction (HMI) device is provided. The electronic device may include circuitry that may be configured to receive touch parameters associated with a physical interaction of the HMI device and a user. Further, the circuitry may be configured to receive control parameters associated with an operator of the HMI device to control the physical interaction of the HMI device and control the physical interaction of the HMI device, based on the received touch parameters and the received control parameters. Further, the circuitry may be configured to determine a physical response of the user, based on the control of the physical interaction of the HMI device, and determine safety metrics associated with the user, based on the determined physical response and the received touch parameters. Further the circuitry may be configured to determine correlation information, based on the control of the physical-interaction of the HMI device and the determined safety metrics, and control the physical-interaction of the HMI device and the user further based on the determined correlation information.

According to another embodiment of the disclosure, a method for safety parameter-based touch-interaction control of human-machine interaction (HMI) device is provided. The method may include receive touch parameters associated with a physical interaction of the HMI device and a user. Further, the method may be configured to receive control parameters associated with an operator of the HMI device to control the physical interaction of the HMI device and control the physical interaction of the HMI device, based on the received touch parameters and the received control parameters. Further, the method may be configured to determine a physical response of the user, based on the control of the physical interaction of the HMI device, and determine safety metrics associated with the user, based on the determined physical response and the received touch parameters. Further, the method may be configured to determine correlation information, based on the control of the physical-interaction of the HMI device and the determined safety metrics, and to control the physical-interaction of the HMI device and the user further based on the determined correlation information.

According to another embodiment of the disclosure, a non-transitory computer-readable medium is provided. The non-transitory computer-readable medium may have stored thereon computer implemented instructions that, when executed by an electronic device, causes the electronic device to execute operations. The operations may include receiving touch parameters associated with a physical-interaction of the HMI device and a user. The operation may further include reception of touch parameters associated with a physical-interaction of the HMI device and a user. The operation may further include reception of control parameters associated with an operator of the HMI device to control the physical-interaction of the HMI device. The operation may further include determination of a physical response of the user, based on the control of the physical interaction of the HMI device, and determination of safety metrics associated with the user, based on the determined physical response and the received touch parameters. The operation may further include determination of correlation information, based on the control of the physical-interaction of the HMI device and the determined safety metrics, and control the physical-interaction of the HMI device and the user further based on the determined correlation information.

### BRIEF DESCRIPTION OF THE DRAWINGS

FIG. 1 is a diagram that illustrates an exemplary network environment for safety parameter-based touch-interaction control of a human-machine interaction (HMI) device, in accordance with an embodiment of the disclosure.

FIG. 2 is a block diagram that illustrates an exemplary electronic device of FIG. 1, for safety parameter-based touch-interaction control of the HMI device, in accordance with an embodiment of the disclosure.

FIG. 3 is a block diagram that illustrates an exemplary HMI device of FIG. 2, for safety parameter-based touch-interaction control of the HMI device, in accordance with an embodiment of the disclosure.

FIG. 4 is a diagram that illustrates a processing pipeline for safety parameter-based touch-interaction control of the HMI device, in accordance with an embodiment of the disclosure.

FIG. 5 is a diagram that illustrates an exemplary physical human-robot touch interaction, in accordance with an embodiment of the disclosure.

FIG. 6 is a flowchart that illustrates operations of an exemplary method for safety parameter-based touch-interaction control of the HMI device, in accordance with an embodiment of the disclosure.

### DETAILED DESCRIPTION

The following described implementation may be found in an electronic device and method for safety parameter-based touch-interaction control of the HMI device. Exemplary aspects of the disclosure may provide an electronic device (for example, a mobile phone, a smart phone, a desktop, a laptop, a personal computer, and the like) that may receive touch parameters (e.g., contact position of the HMI device, speed, force, softness, orientation, etc.) associated with a physical-interaction of the HMI device (e.g., robot) and a user (e.g., patient). Next, the electronic device may receive control parameters (e.g., user-control freedom parameters, transparency factors) associated with an operator of the HMI device to control the physical-interaction of the HMI device. Further, the electronic device may control the physical interaction of the HMI device based on the received touch parameters and the received control parameters. The electronic device may determine a physical response of the user, based on the control of the physical interaction of the HMI device. Also, the electronic device may determine safety metrics associated with the user, based on the determined physical response and the received touch parameters. The electronic device may determine correlation information based on the control of the physical-interaction of the HMI device and the determined safety metrics. Furthermore, the electronic device may control the physical-interaction of the HMI device and the user further based on the determined correlation information.

Typically, dynamics of human-robot interactions (HRI) involves tackling several complex challenges. Effective communication is crucial, yet it is often hindered by the fundamental differences in language processing between humans and robots. Robots must also be capable of predicting and understanding the intricate and often unpredictable nature of human behavior. Long-term interactions add another layer of complexity, requiring robots to continuously adapt to evolving human behaviors and preferences. Prevalent to HRI is the safety of humans, especially in shared environments, along with the protection of their privacy, given the data collected during interactions. The ethical implications of robots' decisions, particularly in ambiguous situations, are also a significant concern. Moreover, establishing metrics for evaluating HRI is essential for consistent advancement and comparison of systems. Lastly, the impact of robots on human social structures and psychological health is an area that demands extensive research. These multifaceted issues underscore the necessity for interdisciplinary collaboration, drawing on expertise from fields such as robotics, AI, psychology, sociology, and design, to enhance the integration of robots into daily human life and activities.

Focusing on a limited set of variables and physiological signals in the context of patient care can have significant implications for both patient safety and comfort. When monitoring is restricted to a few parameters, critical changes in a patient's condition may go unnoticed, potentially leading to adverse events. For instance, relying solely on heart rate and blood pressure without considering other vital signs, like respiratory rate or oxygen saturation, could miss early signs of deterioration. Moreover, the comfort of patients can be affected by the scope of monitoring. Overemphasis on certain signals might lead to unnecessary interventions, causing discomfort or anxiety, while under-monitoring can result in a lack of timely care. It is essential to strike a balance between comprehensive monitoring that ensures safety and selective monitoring that respects patient comfort and avoids alarm fatigue among healthcare providers. While a focused approach to monitoring can be beneficial in certain contexts, it is crucial to consider the broader implications for patient safety and comfort. An integrated approach that accounts for a wide range of physiological signals and environmental factors is key to delivering high-quality patient care. Unlike the most existing methods, a heuristic framework is utilized for a broad spectrum of variables from various angles, aiming for a thorough grasp of human-robot interaction dynamics. It places an innovative emphasis on patient safety and comfort, merging these aspects with adjustable robotic parameters to act as foresightful markers. Diverging from traditional models that concentrate on a restricted array of variables or physiological signals, this approach incorporates a mix of subjective and objective evaluations, including patients' personal reports of comfort and safety as well as measurable metrics of physical movement.

The electronic device of the disclosure may prioritize the human experience, ensuring that any interaction of a human with a robot feels safe and intuitive. For instance, a robot designed to assist patients with mobility must be perceived as safe to gain the confidence of both patients and healthcare staff. The disclosure proposes a model that outlines key factors such as the robot's gentle touch, voice tone, and non-threatening appearance. These factors may be vital to ensure that patients feel comfortable and secure during interactions. Consider a robot designed to assist post-operative patients with walking. The model may indicate that there should be features like padded arms for support, sensors to detect sudden movements indicating a fall, and a reassuring voice to provide instructions and encouragement. By considering patient comfort and robot responsiveness, the robot can be designed to not only assist with physical tasks but also to enhance the overall patient experience by providing a sense of security and companionship.

The electronic device may serve as a comprehensive guide for researchers, engineers, and designers focusing on the human experience, particularly regarding the perception of safety and comfort. It offers an advanced summary of relevant variables for consideration, as well as proposed correlations between these variables and the anticipated outcomes of the system. The recommendations are grounded in insights from physical human-human interactions and in robotic control technologies. For example, based on the physical response of the user safety metrics of the user may be determined. Also, the physical interaction of the HMI device may be controlled based on the safety metrics and the determined control parameters. The method may include a two-step co-design experimental procedure, a user interface that helps users select robot settings, and evaluation tools that measure the interaction experience of the user with the robot using both numbers and words, with an emphasis on safety and comfort. Therefore, both qualitative measures and quantitative measures may be used to enhance user experience of the HMI devices, focusing on perceived safety and comfort. This may further enhance safety and control of the HMI device in performing the relevant action involving an end-user.

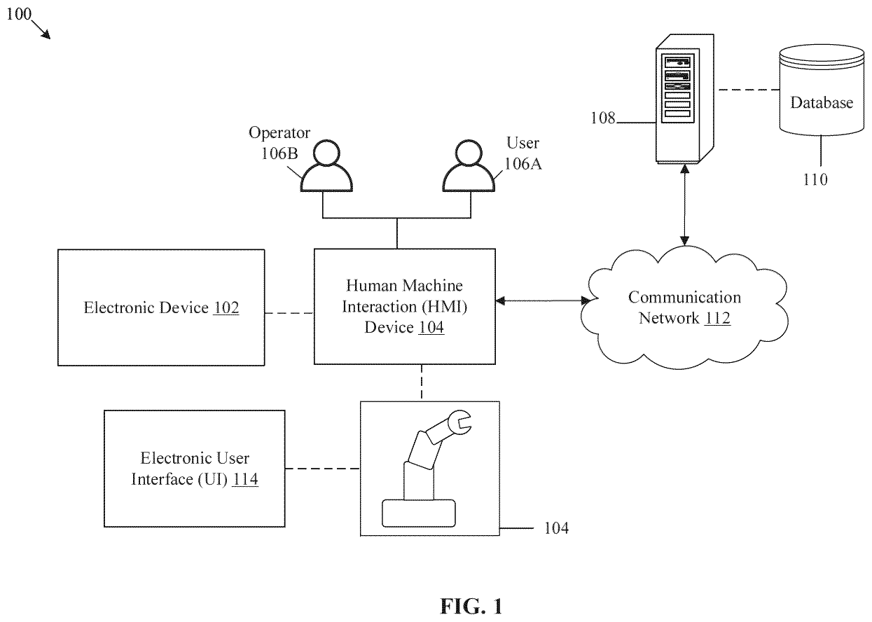

FIG. 1 is a diagram that illustrates an exemplary network environment for safety parameter-based touch-interaction control of a human-machine interaction (HMI) device, in accordance with an embodiment of the disclosure. With reference to FIG. 1, there is shown a network environment 100. The network environment 100 includes an electronic device 102, a human machine interaction (HMI) device 104, a server 108, a database 110, and a communication network 112. The electronic device 102, the HMI device 104, and the server 108 may communicate with one another through one or more networks (such as the communication network 112). The server 108 may be associated with the database 110.

In FIG. 1, there is also shown, a user 106A and an operator 106B associated with (or who may operate) the electronic device 102 and/or the HMI device 104. Though only one user (i.e., the user 106A) and only one operator (i.e., the operator 106B) is shown in FIG. 1, the scope of the disclosure may not be so limited. In some embodiments, the operator 106B may be for example, a remote operator, a nurse, a surgeon, and the like, may be associated with the electronic device 102. Further, the user 106A may be (different from the operator), for example, an end-user, such as a patient, associated with (or may interact with) the HMI device 104. The HMI device 104 may be associated with an electronic user interface (UI) 114. In an example, the electronic UI 114 may be hosted on the HMI device 104. The electronic device 102 may transmit instructions to the HMI device 104 to control the rendering of the electronic UI 114. The electronic device 102 may, additionally or alternatively, be associated with the electronic UI 114.

The electronic device 102 may include suitable logic, circuitry, interfaces, and/or code that may be configured to receive touch parameters (e.g., contact position of the HMI device 104, speed, force, softness, orientation, etc.) and control parameters (e.g., user-control freedom parameters of the HMI device 104, transparency factors of the HMI device 104, etc.). The touch parameters may be associated with a physical-interaction of the HMI device 104 and the user 106A. Further, the control parameters may be associated with the operator 106B to control the physical-interaction. The electronic device 102 may control the physical interaction of the HMI device (e.g., the HMI device 104) based on the received touch parameters and the received control parameters. For example, the physical interaction of the HMI device 104 may include, but is not limited to, physical therapy and rehabilitation, medical care, and physical assistance of the user 106A (such as a patient). The electronic device 102 may determine a physical response of the user 106A, based on the control of the physical interaction of the HMI device (e.g., the HMI device 104). The electronic device 102 may determine safety metrics associated with the user 106A based on the determined physical response and the received touch parameters. The electronic device 102 may determine correlation information based on the control of the physical-interaction of the HMI device and the determined safety metrics. The electronic device 102 may control the physical interaction of the HMI device 104, further based on the determined correlation information. Examples of the electronic device 102 may include, but may not be limited to, a desktop, a tablet, a television (TV), a laptop, a computing device, a smartphone, a cellular phone, a mobile phone, a consumer electronic (CE) device having a display.

The HMI device 104 may include suitable logic, circuitry, interfaces, and/or code that may be configured to perform a predetermined set of actions that may involve a physical interaction of the HMI device 104 and the user 106A, based on touch parameters and control parameters. The operations of the HMI device 104 may be controlled based on the touch parameters and the control parameters. In an example, the HMI device 104 may host an electronic UI (such as the electronic UI 114). The electronic UI 114 may enable reception of the touch parameters and the control parameters. Based on the reception of the various parameters, physical interactions of the HMI device 104 may be controlled. The physical response of the user 106A may be determined based on the control of the physical interaction of the HMI device 104. The HMI device 104 may determine the safety metrics associated with the user 106A based on the physical response and the touch parameters. The HMI device 104 may determine correlation information based on the control of the physical-interaction of the HMI device 104 and the determined safety metrics. Examples of the HMI device 104 may include, but may not be limited to, a robotic arm, a humanoid, a human-interfaced machine, an industrial machine, or any device including software and hardware for touch-based human-machine interaction.

The server 108 that may include suitable logic, circuitry, interfaces, and/or code configured to receive requests from the electronic device 102 to receive parameters from the user 106A. The server 108 may be configured to extract input parameters (e.g., touch parameters and control parameters) associated with the physical interaction of the HMI device 104 and the user 106A. Further, the server 108 may be configured to extract user input indicative of the parameters (e.g., touch parameters and control parameters) based on the control of the rendering of the electronic UI (e.g., electronic UI 114). The server 108 may be configured to control the physical interaction of the HMI device 104 based on the received touch parameters and the received control parameters to determine physical response of the user 106A, based on the control of the physical interaction of the HMI device 104. The safety metrics associated with the user 106A may be determined, based on the physical response and received touch parameters. The server 108 may also extract background parameters associated with the user 106A. The background parameters may include gender of the user 106A, an age of the user 106A, a purpose of touch associated with the HMI device 104, and a physical state of the user 106A, and so on.

The server 108 may execute operations through web applications, cloud applications, HTTP requests, repository operations, file transfer, and the like. Example implementations of the servers 108 may include, but are not limited to, a database server, a file server, a web server, an application server, a mainframe server, a cloud computing server, or a combination thereof. In at least one embodiment, the server 108 may be implemented as a plurality of distributed cloud-based resources by use of several technologies that are well known to those ordinarily skilled in the art. A person with ordinary skill in the art will understand that the scope of the disclosure may not be limited to the implementation of the server 108 and the electronic device 102 as separate entities.

The database 110 may include suitable logic, circuitry, interfaces, and/or code configured to store information such as various parameters (e.g., the touch parameters and the control parameters) received from the user 106A or the operator 106B. Further, the database 110 may store information associated with instructions associated with operation of the HMI device 104. For example, the database 110 may store a mapping table including predefined instructions associated with different values of the parameters to control the operations of the HMI device 104. The database 110 may be derived from data of a relational or non-relational database or a set of comma-separated values (csv) files in conventional or big-data storage. The database 110 may be stored or cached on device or server, such as the server 108. The device storing the database 110 may be configured to query the database 110 for certain information (such as the information related to various parameters (e.g., the touch parameters and the control parameters), information related to the physical response of the user 106A, and the information related to the mapping table) based on reception of a request for the particular information from the electronic device 102. In response, the device storing the database 110 may be configured to retrieve, from the database 110, results (e.g., a user input of the parameters, physical response of the user 106A, and/or the mapping table) based on the received query.

In some embodiments, the database 110 may be hosted on a server 108 located at same or different locations. The operations of the database 110 may be implemented using hardware including a processor, a microprocessor (e.g., to perform or control performance of one or more operations), a field-programmable gate array (FPGA), or an application-specific integrated circuit (ASIC). In some other instances, the database 110 may be implemented using software.

The communication network 112 may include a communication medium through which the electronic device 102, HMI device 104, and the server 108 may communicate with each other. The communication network 112 may be a wired or wireless communication network. Examples of the communication network 112 may include, but are not limited to, Internet, a cloud network, Cellular or Wireless Mobile Network (such as Long-Term Evolution and 5^{th} Generation (5G) New Radio (NR)), satellite communication system (using, for example, low earth orbit satellites), a Wireless Fidelity (Wi-Fi) network, a Personal Area Network (PAN), a Local Area Network (LAN), or a Metropolitan Area Network (MAN). Various devices in the network environment 100 may be configured to connect to the communication network 112, in accordance with various wired and wireless communication protocols. Examples of such wired and wireless communication protocols may include, but are not limited to, at least one of a Transmission Control Protocol and Internet Protocol (TCP/IP), User Datagram Protocol (UDP), Hypertext Transfer Protocol (HTTP), File Transfer Protocol (FTP), Zig Bee, EDGE, IEEE 802.11, light fidelity (Li-Fi), 802.16, IEEE 802.11s, IEEE 802.11g, multi-hop communication, wireless access point (AP), device to device communication, cellular communication protocols, and Bluetooth (BT) communication protocols.

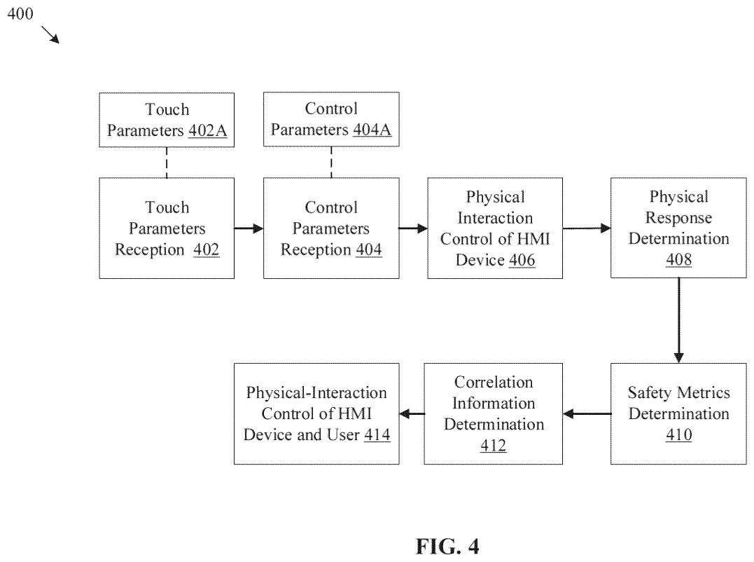

In operation, the electronic device 102 may be configured to enhance the safety and comfort of the user 106A in physical human-robot interaction by integrating the touch parameters associated with the user 106A with the control parameters associated with the operator 106B of the HMI device 104. The electronic device 102 may be configured to receive the touch parameters associated with a physical-interaction of the HMI device 104 and the user 106A. In an embodiment, the touch parameters may include, but are not limited to, a contact position of the HMI device 104 on a body portion of the user 106A, a speed of the HMI device, a force of the HMI device, a softness of the HMI device, and an orientation of the HMI device, and so on. The reception of the touch parameters is described further, for example, in FIG. 4 (at 402).

The electronic device 102 may be configured to receive the control parameters associated with the operator 106B of the HMI device 104 to control the physical-interaction of the HMI device 104. In an embodiment, the control parameters may include, but are not limited to, a user-control freedom parameter of the HMI device 104 for the operator 106B or a transparency factor of the HMI device for the operator, etc. The reception of the control parameters is described further, for example, in FIG. 4 (at 404).

The electronic device 102 may be configured to control the physical interaction of the HMI device 104 based on the touch parameters and the control parameters. For example, the electronic device 102 may determine set of instructions associated with the physical interaction, based on the received touch parameters and the control parameters. Thereafter, the electronic device 102 may transmit the determined set of instructions to the HMI device 104 to control an operation of the HMI device 104 and thereby control the physical interaction of the HMI device 104 with the user 106A. The control of the physical interaction is described further, for example, in FIG. 4 (at 406).

The electronic device 102 may be configured to determine the physical response of the user 106A, based on the control of the physical interaction of the HMI device (e.g., the HMI device 104). The physical response of the user 106A may include, but is not limited to, a gaze of the user 106A, a facial expression of the user 106A, and involuntary motion of the user 106A, etc. The determination of the physical response of the user is described further, for example, in FIG. 4 (at 408).

The electronic device 102 may be configured to determine the safety metrics associated with the user 106A, based on the determined physical response and the touch parameters. The safety metrics may include, but is not limited to, a trust level of the user associated with the HMI device 104, a comfort level of the user 106A associated with the HMI device 104, and a safety level of the user 106A associated with the HMI device 104, etc. The determination of the safety metrics is described further, for example, in FIG. 4 (at 410).

The electronic device 102 may be configured to determine the correlation information based on the control of the physical-interaction of the HMI device 104 and the determined safety metrics. For example, the electronic device 102 may transmit instructions to the HMI device to control the physical interaction of the HMI device. The electronic device 102 may determine the safety metrics based on the physical response of the user 106A (received based on the physical interaction between the user 106A and the HMI device 104). The electronic device 102 may determine the correlation information based on the control of the physical-interaction and the safety metrics. The determination of the correlation information is described further, for example, in FIG. 4 (at 412).

The electronic device 102 may control the physical interaction of the HMI device 104, based on the determined correlation information. The electronic device 102 may transmit instructions to the HMI device 104 to control the physical interaction further based on the correlation information. The control of the physical interaction of the HMI device is described further, for example, in FIG. 4 (at 414).

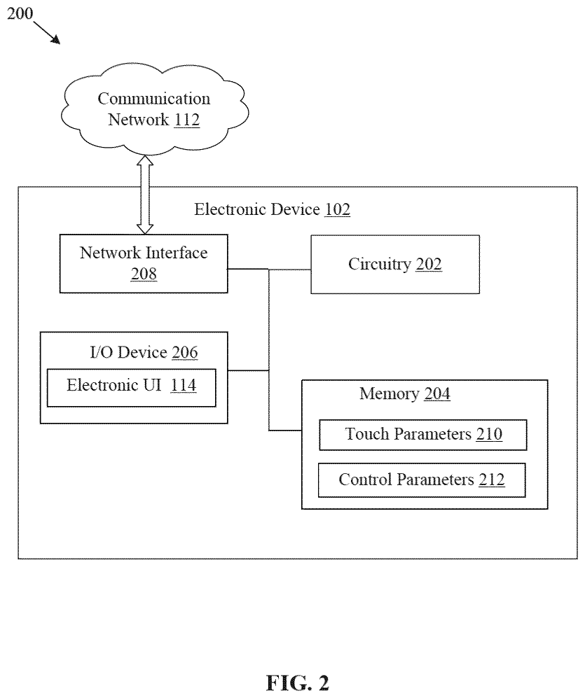

FIG. 2 is a block diagram that illustrates an exemplary electronic device of FIG. 1, for safety parameter-based touch-interaction control of the HMI device, in accordance with an embodiment of the disclosure. FIG. 2 is explained in conjunction with elements from FIG. 1. With reference to FIG. 2, there is shown a block diagram 200 of the electronic device 102. The electronic device 102 may include a circuitry 202, a memory 204, an input/output (I/O) device 206, and a network interface 208. In at least one embodiment, the I/O device 206 may also include the electronic user interface (UI) 114. In at least one embodiment, the memory 204 may include parameters (e.g., touch parameters 210 and control parameters 212). The circuitry 202 may be communicatively coupled to the memory 204, the I/O device 206, the network interface 208, through wired or wireless communication of the electronic device 102.

The circuitry 202 may include suitable logic, circuitry, and interfaces that may be configured to execute program instructions associated with different operations to be executed by the electronic device 102. The operations may include controlling the physical interaction of the HMI device 104 based on the received touch parameters 210 and the received control parameters 212. The operations may further include determination of the physical response of the user 106A, determination of the safety metrics, and determination of correlation information based on the control of the physical interaction and the safety metrics.

The circuitry 202 may include one or more specialized processing units, which may be implemented as an integrated processor or a cluster of processors that perform the functions of the one or more specialized processing units, collectively. The circuitry 202 may be implemented based on a number of processor technologies known in the art. Examples of implementations of the circuitry 202 may be an x86-based processor, a Graphics Processing Unit (GPU), a Reduced Instruction Set Computing (RISC) processor, an Application-Specific Integrated Circuit (ASIC) processor, a Complex Instruction Set Computing (CISC) processor, a microcontroller, a central processing unit (CPU), and/or other computing circuits.

The memory 204 may include suitable logic, circuitry, interfaces, and/or code that may be configured to store the program instructions to be executed by the circuitry 202. The program instructions stored on the memory 204 may enable the circuitry 202 to execute operations of the circuitry 202 (and/or the electronic device 102). In at least one embodiment, the memory 204 may store the parameters (e.g., the touch parameters 210 and the control parameters 212). The touch parameters 210 may include the contact position, speed, force, softness, orientation, and so on. The control parameters 212 may include the user-control freedom parameters, the transparency factors, and the like. Further, the memory 204 may store information about the physical response of the user 106A and the correlation information. In an embodiment, the physical response of the user 106A may include the body movement, the gaze, the verbal response and the facial response of the user 106A, and the like. Further, the correlation information may be determined based on the physical response of the user 106A and the touch parameters 210 between the user 106A and the HMI device 104. Examples of implementation of the memory 204 may include, but are not limited to, Random Access Memory (RAM), Read Only Memory (ROM), Electrically Erasable Programmable Read-Only Memory (EEPROM), Hard Disk Drive (HDD), a Solid-State Drive (SSD), a CPU cache, and/or a Secure Digital (SD) card.

The I/O device 206 may include suitable logic, circuitry, interfaces, and/or code that may be configured to receive an input and provide an output based on the received input. For example, the I/O device 206 may receive a user input from the user 106A or the operator 106B. The reception of various parameters (e.g., the touch parameters 210, the control parameters 212, and the like) may be indicative of operating parameters for the HMI device 104. In some embodiments, the I/O device 206 may receive the touch parameters 210 and the control parameters 212. Examples of the I/O device 206 may include, but are not limited to, a touch screen, a keyboard, a mouse, a joystick, a microphone, the display device 102B, and a speaker. Examples of the I/O device 206 may further include braille I/O devices, such as, braille keyboards and braille readers.

The I/O device 206 may include the electronic UI 114. The electronic UI 114 may include suitable logic, circuitry, and interfaces that may be configured to receive instructions from the circuitry 202 to render parameters (e.g., the touch parameters 210 and the control parameters 212, and the like) on a display screen. In at least one embodiment, the electronic UI 114 may be at least one of a resistive touch screen, a capacitive touch screen, or a thermal touch screen. The electronic UI 114 may be realized through several known technologies such as, but not limited to, at least one of a Liquid Crystal Display (LCD) display, a Light Emitting Diode (LED) display, a plasma display, or an Organic LED (OLED) display technology, or other display devices.

The network interface 208 may include suitable logic, circuitry, and interfaces that may be configured to facilitate communication between the electronic device 102, the HMI device 104, and the server 108, via the communication network 112. The network interface 208 may be implemented by use of various known technologies to support wired or wireless communication of the electronic device 102 with the communication network 112. The network interface 208 may include, but is not limited to, an antenna, a radio frequency (RF) transceiver, one or more amplifiers, a tuner, one or more oscillators, a digital signal processor, a coder-decoder (CODEC) chipset, a subscriber identity module (SIM) card, or a local buffer circuitry.

The network interface 208 may be configured to communicate via wireless communication with networks, such as the Internet, an Intranet, or a wireless network, such as a cellular telephone network, a wireless local area network (LAN), a short-range communication network, and a metropolitan area network (MAN). The wireless communication may use one or more of a plurality of communication standards, protocols and technologies, such as Global System for Mobile Communications (GSM), Enhanced Data GSM Environment (EDGE), wideband code division multiple access (W-CDMA), Long Term Evolution (LTE), 5^{th} Generation (5G) New Radio (NR), code division multiple access (CDMA), time division multiple access (TDMA), Bluetooth, Wireless Fidelity (Wi-Fi) (such as IEEE 802.11a, IEEE 802.11b, IEEE 802.11g or IEEE 802.11n), voice over Internet Protocol (VoIP), light fidelity (Li-Fi), Worldwide Interoperability for Microwave Access (Wi-MAX), a near field communication protocol, and a wireless pear-to-pear protocol.

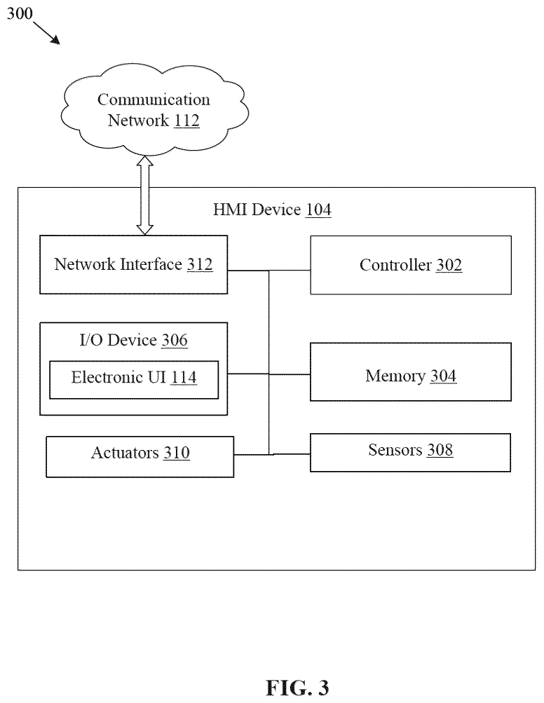

FIG. 3 is a block diagram that illustrates an exemplary HMI device of FIG. 1, for safety parameter-based touch-interaction control of the HMI device, in accordance with an embodiment of the disclosure. FIG. 3 is explained in conjunction with elements from FIGS. 1 and 2. With reference to FIG. 3, there is shown a block diagram 300 of the HMI device 104. The HMI device 104 may include controller 302, a memory 304, an input/output (I/O) device 306, sensors 308, actuators 310, and network interface 312. In at least one embodiment, the I/O device 306 may also include the electronic UI 114. The controller 302 may be communicatively coupled to the memory 304, the I/O device 306, the network interface 312, the sensors 308, and actuators 310 through wired or wireless communication of the HMI device 104.

The controller 302 may include suitable logic, circuitry, and interfaces that may be configured to execute program instructions associated with different operations to be executed by the HMI device 104. The controller 302 may be a computer system that connects to the HMI device 104 in order to control the physical interaction. In addition to physical interaction, the controller 302 may also be responsible for end-effector and to prevent interference from occurring within the environment. Robotic programs may be coded into the controller 302, which may be the electronic device 102 that may consist of buttons, switches, or a touchscreen to allow for the input of programming commands.

The controller 302 may include one or more specialized processing units, which may be implemented as an integrated processor or a cluster of processors that perform the functions of the one or more specialized processing units, collectively. The controller 302 may be implemented based on a number of processor technologies known in the art. Examples of implementations of the controller 302 may be an x86-based processor, a Graphics Processing Unit (GPU), a Reduced Instruction Set Computing (RISC) processor, an Application-Specific Integrated Circuit (ASIC) processor, a Complex Instruction Set Computing (CISC) processor, a microcontroller, a central processing unit (CPU), and/or other computing circuits.

The memory 304 may include suitable logic, circuitry, interfaces, and/or code that may be configured to store the program instructions to be executed by the controller 302. The program instructions stored on the memory 304 may enable the controller 302 to execute operations of the controller 302 (and/or the HMI device 104). In at least one embodiment, the memory 304 may store the parameters (e.g., the touch parameters 210 and the control parameters 212). The memory 304 may further store values of the touch parameters, the control parameters, and the information about the physical response of the user 106A, and the like. Examples of implementation of the memory 304 may include, but are not limited to, Random Access Memory (RAM), Read Only Memory (ROM), Electrically Erasable Programmable Read-Only Memory (EEPROM), Hard Disk Drive (HDD), a Solid-State Drive (SSD), a CPU cache, and/or a Secure Digital (SD) card.

The I/O device 306 may include suitable logic, circuitry, interfaces, and/or code that may be configured to receive an input and provide an output based on the received input. For example, the I/O device 306 may receive a user input from the user 106A or the operator 106B. The user input may be indicative of parameters (e.g., the touch parameters 210, the control parameters 212, and the like) for the HMI device 104. In some embodiments, the I/O device 306 may receive input from the user 106A that may be indicative of the parameters based on the control of the rendering of the electronic UI 114. Examples of the I/O device 306 may include, but are not limited to, a touch screen, a keyboard, a mouse, a joystick, a microphone, the display device, and a speaker. Examples of the I/O device 306 may further include braille I/O devices, such as braille keyboards and braille readers.

The I/O device 306 may include the electronic UI 114. The electronic UI 114 may include suitable logic, circuitry, and interfaces that may be configured to receive instructions from the controller 302 to render, on a display screen, UI elements (e.g., first UI elements, second UI elements, etc.), the information about the physical response of the user 106A. In at least one embodiment, the electronic UI 114 may be at least one of a resistive touch screen, a capacitive touch screen, or a thermal touch screen. The electronic UI 114 may be realized through several known technologies such as, but not limited to, at least one of a Liquid Crystal Display (LCD) display, a Light Emitting Diode (LED) display, a plasma display, or an Organic LED (OLED) display technology, or other display devices.

The actuators 310 may receive signals from the controller 302 and execute the corresponding physical movement. There may be different type of actuators 310 used in the HMI device 104, depending on the load associated with factors such as, but not limited to force, torque, speed of operation, precision, accuracy, and power consumption. The actuators 310 may for example receive input from the user 106A/operator 106B to control the operation of the HMI device 104 and the actuator 310 may be operated based on the control command received from the controller 302.

The sensors 308 may measure some attribute of their environment and convert it into a signal that can be read or interpreted by the HMI device 104. The sensors 308 may help robots to determine and measure the geometric and physical properties of objects in their surrounding environment, such as position, orientation, velocity, acceleration, distance, size, force, moment, temperature, luminance, weight, etc. The sensors 308 may be essential for robots to operate with high precision and efficiency, and to interact safely and effectively with their environment and with other machines. The sensors 308 used in the HMI device 104 may include, but not limited to proprioceptive sensors, exteroceptive sensors, light sensors, and sound sensors.

The network interface 312 may include suitable logic, circuitry, and interfaces that may be configured to facilitate communication between the controller 302, the I/O device 306, and the memory 304, via the communication network 112. The network interface 312 may be implemented by use of various known technologies to support wired or wireless communication of the HMI device 104 with the communication network 112. The network interface 312 may include, but is not limited to, an antenna, a radio frequency (RF) transceiver, one or more amplifiers, a tuner, one or more oscillators, a digital signal processor, a coder-decoder (CODEC) chipset, a subscriber identity module (SIM) card, or a local buffer circuitry.

The network interface 312 may be configured to communicate via wireless communication with networks, such as the Internet, an Intranet, or a wireless network, such as a cellular telephone network, a wireless local area network (LAN), a short-range communication network 312, and a metropolitan area network (MAN). The wireless communication may use one or more of a plurality of communication standards, protocols and technologies, such as Global System for Mobile Communications (GSM), Enhanced Data GSM Environment (EDGE), wideband code division multiple access (W-CDMA), Long Term Evolution (LTE), 5^{th} Generation (5G) New Radio (NR), code division multiple access (CDMA), time division multiple access (TDMA), Bluetooth, Wireless Fidelity (Wi-Fi) (such as IEEE 802.11a, IEEE 802.11b, IEEE 802.11g or IEEE 802.11n), voice over Internet Protocol (VoIP), light fidelity (Li-Fi), Worldwide Interoperability for Microwave Access (Wi-MAX), a near field communication protocol, and a wireless pear-to-pear protocol.

FIG. 4 is a diagram that illustrates a processing pipeline for safety parameter-based touch-interaction control of the HMI device, in accordance with an embodiment of the disclosure. With reference to FIG. 4, there is shown an exemplary execution pipeline 400 for safety parameter-based touch-interaction control of the HMI device 104. The execution pipeline 400 may include operations 402 to 414 executed by a computing device, such as, the electronic device 102 of FIG. 1 or the circuitry 202 of FIG. 2.

At 402, an operation for reception of touch parameters may be executed. The circuitry 202 may be configured to receive touch parameters 402A associated with the

HMI device 104 and the user 106A. The touch parameters 402A may include, but are not limited to, a contact position (e.g., push buttons, toggle switches, selector switches, and the like) of the HMI device 104, a speed (m/s), a force (N), a softness, and an orientation (X, Y, Z and the like). In an example scenario, the received touch parameters may indicate that the HMI device 104 may be required to apply 1 N of force, for 1 minute duration, at a certain body portion of the user 106A. The orientation of the HMI device 104 may use a combination of rotations around the X, Y, and Z axes to achieve a desired orientation. This could be represented by a rotation matrix or a rotation vector in the robot's control system. Additionally, the HMI device 104, such as collaborative robots, may use an orientation vector based on the axis-angle representation, which involves rotating around a specific vector by a certain angle. Another example is the use of RPY (roll, pitch, yaw) values that are often used in the interfaces of robots, which uses a ZY′X″ convention for Euler angles. In an embodiment, the contact position may refer to the area where physical interaction occurs between the HMI device 104 and user 106A. An example of a contact zone may be a scenario where a collaborative HMI device and the user 106A work together to carry a heavy object. The collaborative HMI device and the user 106A may include sensors to detect the user's presence and apply the appropriate force to assist with the lifting task, ensuring safety and efficiency in the shared workspace. Another example is the use of dynamic safety zones in industrial settings, where a safety-certified camera monitors the distance between the HMI device 104 and the user 106A. If the user 106A enters a predefined detection zone, the HMI device 104 may perform a safety-rated monitored stop to prevent accidents.

At 404, an operation for reception of the control parameters may be executed. The circuitry 202 may be configured to receive the control parameters 404A associated with the operator 106B of the HMI device 104 to control the physical-interaction of the HMI device 104 with a user (e.g., the user 106A). The control parameters 404A associated with the operator 106B may include, but are not limited to, a user-control freedom parameter of the HMI device 104 for the operator 106B and a transparency factor of the HMI device 104 for the operator 106B. The user-control freedom parameters of the HMI device 104 may refer to the various degrees of freedom for controlling the HMI device 104. Such parameters define the flexibility and range of motion that the robot can achieve under human control, for example, degrees of freedom (DOF), force/torque sensing, velocity and position control, admittance and impedance control, and the like. The transparency factors of the HMI device 104 may refer to communicative indicators for controlling the HMI device 104 to ensure the predictability of the actions and next moves of the HMI device for the user 106A. Such parameters may include, but are not limited to, incorporating communicative behaviors such as verbally notifying the touch behaviors prior to touch activities; sound or visual alerts indicating actions before they begin or when they are completed; and/or physically demonstrating the behaviors before performing the actual touch interactions. The transparency factor and predictability in robot actions may be crucial for enhancing human trust and safety perception in the HMI devices 104. The transparent HMI device works, where the robot's behaviors and decision-making processes are clear, may allow humans to understand and predict the robot's next moves, leading to a sense of control and safety. Predictable robots, which behave consistently and reliably, enable humans to form accurate mental models of robot behavior, facilitating smoother collaboration. This predictability in robot actions is essential, as it reduces the fear of unexpected behaviors and fosters an environment where humans feel safe to rely on robots especially in critical tasks.

At 406, an operation for the physical interaction control may be executed. The circuitry 202 of the electronic device 102 may be configured to control the physical interaction of the HMI device 104 based on the received touch parameters 210 and the received control parameters 212. The circuitry 202 may determine instructions to control the physical interaction of the HMI device 104 based on the received touch parameters 210 and the received control parameters 212. In an example scenario, the received touch parameters 210 may indicate that the HMI device 104 may be required to apply 1 N of force, for 1 minute duration, at a certain body portion of the user 106A. Further, the received control parameters 212 may indicate that the HMI device 104 may be required to enable the operator 106B to manually control the HMI device 104 across at least 3 degrees of freedom at a time. Accordingly, the circuitry 202 may determine the corresponding instructions to control the HMI device 104. The circuitry 202 may also transmit the determined instructions to the HMI device 104. In one embodiment, the HMI device 104 may look-up an instruction table in the memory 304 to interpret the instructions and convert the interpreted instructions to corresponding control commands. In other embodiments, the circuitry 202 may convert the instructions to corresponding control commands and transmit the control commands to the HMI device 104. The HMI device 104 may execute the corresponding control commands and the various components of the HMI device 104 may be accordingly controlled. Also, the physical interaction of the HMI device 104 may be further controlled based on the correlation information, as described further, for example, at 414.

At 408, an operation for determination of physical response of the user may be executed. The circuitry 202 may be configured to determine the physical response of the user 106A, based on the control of the physical interaction of the HMI device 104. The physical response of the user 106A may be determined based on the control of the physical interaction of the HMI device 104. The physical response of the user 106A may include, but is not limited to, a body movement of the user 106A, a gaze of the user 106A, a verbal response of the user 106A, and a facial response of the user 106A. In one embodiment, the controller 302 may determine the physical response of the user 106A, for example, based on use of the sensors 308. The circuitry 202 may receive information related to the determined physical response of the user 106A from the controller 302. In addition, or alternatively, the electronic device 102 may include sensors (not shown in FIG. 2), such as an image capture device, a microphone, and the like. In such a case, the circuitry 202 may control the sensors of the electronic device 102 to determine the physical response of the user 106A. The HMI device 104 may be configured to operate based on a co-design experimental procedure with at least two iterations, a user interface that may help users to select robot settings, and evaluation tools that may measure the interaction experience of the users with the robot using both numbers and words with an emphasis on safety and comfort. Therefore, both qualitative measures and quantitative measures may be used to enhance user experience of the HMI devices focusing on perceived safety and comfort. The safety and control of the HMI device 104 in performing the relevant action involving an end-user may be enhanced based on the user experience and physical response based iterative control of the HMI device 104.

At 410, an operation for determination of safety metrics associated with the user may be executed. The circuitry 202 may be configured to determine the safety metrics associated with the user 106A, based on the determined physical response and the received touch parameters 210. The safety metrics may include, but are not limited to, the trust level of the user 106A associated with the HMI device 104, the comfort level of the user 106A associated with the HMI device 104, and the safety level of the user 106A associated with the HMI device 104. In an example scenario, the physical response of the user 106A may indicate that the user 106A has relaxed facial expressions, and the touch parameters 210 may indicate that the HMI device 104 may have applied a force of 1 N, for 1 minute, at an arm region of the user 106A to sooth a pain of the user 106A. In this scenario, the circuitry 202 may determine that the safety metrics level of the user 106A may be high (e.g., close to “1”, assuming that the safety metrics level ranges from “0” to “1”, where “1” is the highest and “0” is the lowest).

At 412, an operation of determination for the correlation information may be executed. The circuitry 202 may be configured to determine the correlation information based on the control of the physical-interaction of the HMI device 104 and the determined safety metrics. The correlation information may be determined based on an inter-relation of the control of the physical-interaction and the determined safety metrics. The circuitry 202 may determine a relationship between the physical-interaction control and the safety metrics. The relationship between the physical-interaction control and the safety metrics may correspond to the correlation information. For example, the physical-interaction of the HMI device 104 may be controlled based on the received touch parameters 210 and the control parameters 212. In an example scenario, the HMI device 104 may be designed to assist users in regaining mobility after a stroke or injury. The HMI device 104 may be equipped with various sensors and actuators that respond to the touch parameters 210 and the control parameters 212. Parameters such as range of motion, resistance level, and repetition count may be provided as input to the HMI device 104. As the user 106A moves, the sensors may detect the limb's position, velocity, and force, which may also be displayed in real-time on the electronic device 102 for both the user 106A and the operator 106B to monitor. If the user 106A experiences discomfort, the user 106A or the operator 106B may touch a ‘Decrease Intensity’ button on the electronic UI 114 of the electronic device 102, and the HMI device 104 may adjust settings of the HMI device 104 to reduce the resistance. Throughout the session, the operator 106B may use the HMI device to fine-tune the control parameters 212 based on the user interaction and physical response of the user, enabling a personalized and adaptive rehabilitation process. In a physiotherapy rehabilitation example with a robotic exoskeleton, safety metrics may involve the precision of the movements of the HMI device 104, the frequency of malfunctions, and the physical response of the user 106A on pain or discomfort. The ability of the HMI device 104 to translate the touch parameters 210 into precise values of the control parameters 212 for the HMI device 104 operation ensures that the device assists the patient's movements accurately and in a comfortable, safe manner. The circuitry 202 may capture such inter-relationship between the control of the physical-interaction and the safety metrics as the correlation information.

At 414, the operation of the control of physical interaction of the HMI device 104 may be executed. The circuitry 202 of the electronic device 102 may be configured to control the physical-interaction of the HMI device 104 and the user 106A, further based on the determined correlation information. For example, the circuitry 202 may determine instructions to control the physical interaction of the HMI device 104 based on the received touch parameters 210, the received control parameters 212, and determined correlation information. In one embodiment, the circuity 202 may transmit the determined instructions to the HMI device 104, and the HMI device 104 may convert the determined instructions to corresponding control commands. In other embodiments, the circuitry 202 may convert the determined instructions to corresponding control commands and transmit the control commands to the HMI device 104. The HMI device 104 may execute the corresponding control commands and the various components of the HMI device 104 may be accordingly controlled. In an example scenario, the user background parameters (e.g., the gender, age and physical state of the user 106A, a purpose of touch associated with the HMI device 104, etc.) may be used while performing the robot-user interaction. For example, a behavior of the HMI device 104 may be configured based on the user-background parameters. In a case of aged users, the HMI device 104 may apply low amount of force and may apply the force on smaller portions of the body to ensure comfort for the aged user. The correlation information for aged users may be used to determine the amount of force and a size of body portion of the aged user that ensures comfort and safety for the aged user (based on the safety metrics). The physical interaction of the HMI device 104 may be controlled based on the touch parameters 210, control parameters 212, and correlation information.

The electronic device 102 of the disclosure may enable a human-robot interaction to gather insights and specify robot design choices when investigating physical human-robot interaction scenarios, especially in the tele-operated health care that involve instrumental touch interactions. In essence, the outcomes of HRI may be pivotal for those creating future tele-operated healthcare robots. It emphasizes the importance of understanding the nuances of physical interactions between humans and robots to design systems that are effective, safe, and user-friendly. This may be particularly relevant in healthcare settings where precision and reliability are critical.

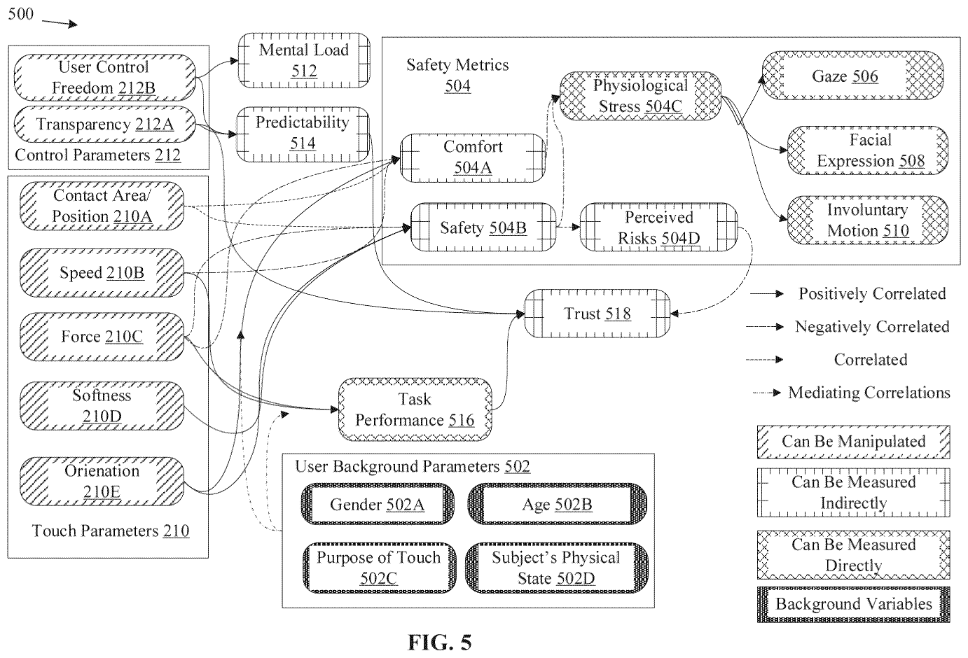

FIG. 5 is a diagram that illustrates an exemplary scenario for physical human-robot touch interaction, in accordance with an embodiment of the disclosure. FIG. 5 is explained in conjunction with elements from FIGS. 1, 2, 3 and 4. With reference to FIG. 5, there is shown an exemplary scenario 500 of a physical human-robot touch interaction. The scenario 500 may include various parameters, which may affect determination of a safety and comfort perspective of the user 106A. The various parameters may include for example, the touch parameters 210, the control parameters 212, a user's background parameters 502, safety metrics 504 of the user 106A, mental load 512, a predictability 514, a task performance 516, and a trust 518. The touch parameters 210 may include, but are not limited to, a contact area/position 210A, a speed 210B, a force 210C, a softness 210D, and an orientation 210E. The control parameters 212 may include, but are not limited to, a user control freedom 212B, and a transparency 212A. The user's background parameters 502 may include, but are not limited to, a gender 502A, an age 502B, a purpose of touch 502C, and a subject's physical state 502D. The safety metrics 504 may include, but are not limited to, a comfort 504A, a safety 504B, a physiological stress 504C, a perceived risks 504D, a gaze 506, a facial expression 508, and an involuntary motion 510.

The user control freedom 212B may be positively correlated to mental load 512, and the predictability 514. The transparency 212A of the control parameters 212 may be positively correlated to the predictability 514 and the trust 518. The contact area/position 210A of the HMI device 104 may be correlated to the comfort 504A and safety 504B of the safety metrics 504. The speed 210B may be negatively correlated to the safety 504B. The speed 210B may be positively correlated to the task performance 516. The force 210C may be negatively correlated to the comfort 504A and the safety 504B. The force 210C may be positively correlated to the task performance 516. The softness 210D may be positively correlated to the safety 504B. The orientation 210E may be positively correlated to the safety 504B and comfort 504A.

The comfort 504A may be negatively correlated to the physiological stress 504C. The safety 504B may be negatively correlated with the physiological stress 504C and the perceived risks 504D. The task performance 516 may be positively correlated with the trust 518. The perceived risks 504D may be negatively correlated to the trust 518. The physiological stress 504C may be correlated with the gaze 506, facial expression 508, and involuntary motion 510. Further, the user's background parameters 502 may act as mediated parameters for the various parameters, for instance, but not limited to, the comfort 504A and the task performance 516.

In an embodiment, the parameters may be manipulated, measured directly, measured indirectly, or background variables. The parameters that may be manipulated include, for example, the control parameters 221 (e.g., the user control freedom 212B, the transparency 212A) and the touch parameters 210 (e.g., the contact area/position 210A, the speed 210B, the force 210C, softness 210D, and the orientation 210E). The parameters that may be measured directly include, for example, the task performance 516, the physiological stress 504C, the gaze 506, the facial expression 508, and the involuntary motion 510. The parameters that may be measured indirectly include, for example, the mental load 512, the predictability 514, the comfort 504A, the perceived risks 504D, and the trust 518. The parameters that may be background variables include user background parameters 502, for example, the gender 502A, the age 502B, the purpose of touch 502C, and the subject's physical state 502D. It should be noted that the scenario 500 of FIG. 5 is for exemplary purposes and should not be construed to limit the scope of the disclosure.

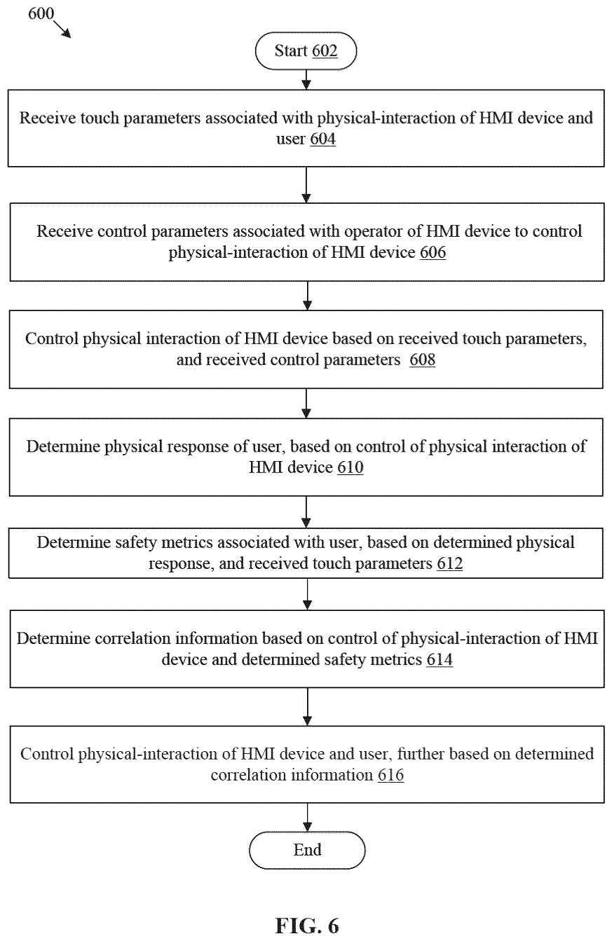

FIG. 6 is a flowchart that illustrates operations of an exemplary method for safety parameter-based touch-interaction control of the HMI device, in accordance with an embodiment of the disclosure. FIG. 6 is explained in conjunction with elements from FIGS. 1, 2, 3, 4, and 5. With reference to FIG. 6, there is shown a flowchart 600. The flowchart 600 may include operations from 602 to 616 and may be implemented by the electronic device 102 of FIG. 1. The flowchart 600 may start at 602 and proceed to 604.

At 604, the touch parameters associated with the physical-interaction of the HMI device 104 and user 106A may be received. The circuitry 202 may be configured to receive the touch parameters 210 associated with the physical-interaction of the HMI device and the user 106A. The touch parameters 210 may include, but not limited to, the contact position, speed. force, softness, orientation, and so on. The reception of touch parameters is described further, for example, in FIG. 4 (at 402).

At 606, the control parameters associated with the operator 106B and the HMI device 104 may be received to control the physical interaction of the HMI device 104. The circuitry 202 may be configured to receive the control parameters 212 associated with the operator 106B of the HMI device 104 to control the physical-interaction of the HMI device 104. The control parameters 212 associated with the operator 106B include, but not limited to, user-control freedom parameters of the HMI device 104 for the operator 106B and transparency factor of the HMI device 104 for the operator 106B. The user-control freedom parameters in the HMI device 104 may refer to the various degrees of freedom (DOF) that an operator 106B has when controlling the HMI device 104. The transparency factors of the HMI device 104 may refer to various communicative indicators that an operator 106B has when controlling the HMI device 104 to ensure the predictability of the actions and next moves of the HMI device for the user 106A. The reception of control parameters is described further, for example, in FIG. 4 (at 404).

At 608, the physical interaction of the HMI device 104 may be controlled based on received touch parameters 210 and received control parameters 212. The circuitry 202 may be configured to control the physical interaction of the HMI device 104 based on the received touch parameters 210 and the received control parameters 212. The physical interaction using parameters such as, but not limited to, the touch parameters 210 and the control parameters 212, may involve understanding factors influencing movement and interactions of the HMI device 104. For instance, the HMI device 104 may be programmed to respond to the physical human interventions by adjusting its trajectory. The parameters may be set by the user 106A or the operator 106B.

In an embodiment, the variety of HMI devices 104 include robots and interfaces, depending on the control and programming approach, the environment and task, and the human-machine interaction. For example, some robots may use collaborative robots that can perform complex tasks in various environments, while others may use manual robots that require complete human intervention for their operation. Some interfaces may use a robot operating system (ROS), which may be a framework that provides a painless entry point for nonprofessionals in the field of programming robots. The control of the physical interaction of the HMI device is described further, for example, in FIG. 4 (at 406).

At 610, the physical response of the user may be determined, based on the control of the physical interaction of the HMI device 104. The circuitry 202 may be configured to determine the physical response of the user 106A, based on the control of the physical interaction of the HMI device 104. The physical response may be determined based on tracking of user inputs, system responses, and the overall performance of the interaction process. To effectively record the physical response of the user 106A, continuous user feedback may be essential for refining the HMI device 104. For example, the electronic device 102 and/or HMI device 104 may include sensors, such as image capture devices, to capture images of the user 106A, while the HMI device 104 is interacting with the user 106A. Based on the captured images, a physical response of the user 106A, such as the facial expressions and body movement of the user 106A, may be determined. The determination of the physical response of the user is described further, for example, in FIG. 4 (at 408).

At 612, the safety metrics associated with the user may be determined, based on the physical response and the received touch parameters. The circuitry 202 may be configured to determine the safety metrics associated with the user 106A, based on the determined physical response and the received touch parameters 210. The safety metrics in human-robot interaction (HRI) are quantitative measures used to assess and enable the safety of humans when they are in close proximity to or interacting with robots. These metrics are important in environments where robots and humans coexist, such as manufacturing floors, healthcare facilities, and even homes. For example, if the user 106A has a facial expression that indicates that the user 106A is comfortable and the HMI device 104 is a portable hand-held device that applies a small amount of force on a body portion of the user 106A to alleviate a pain of the user 106A, the safety metrics level may have a high value (e.g., a value close to “1”). The determination of the safety metrics associated with the user is described further, for example, in FIG. 4 (at 410).

At 614, correlation information may be determined based on the control of the physical-interaction of the HMI device and the determined safety metrics. The circuitry may be configured to determine correlation information, based on the control of the physical-interaction of the HMI device 104 and the determined safety metrics. The safety metrics may include but not limited to the trust level of the user 106A associated with the HMI device 104, the comfort level of the user 106A associated with the HMI device 104, and the safety level of the user 106A associated with the HMI device 104. For example, a robotic arm is used to assist the patient in performing specific movements. The HMI device 104 may provide a controlled environment where the physical interaction may be precisely measured and adjusted according to the patient's needs. In an example scenario, therapists may collect data on these safety metrics and analyze the correlation between the robot's physical interaction and the patient's recovery progress. For example, they may find that a certain level of force applied by the robot correlates with better accuracy of movement in the patient's limb, leading to more effective therapy sessions. The determination of the correlation information associated with the user is described further, for example, in FIG. 4 (at 412).

At 616, the physical interaction of the HMI device may be controlled, further based on the determined correlation information. The circuitry 202 may be configured to control the physical interaction of the HMI device 104 based on the determined correlation information. In an example of healthcare, robots and user interactions may be observed, and these robots may assist users (e.g., the user 106A) who are recovering from injuries or surgeries. For instance, a robotic arm may help a patient perform physical therapy exercises. The robot may adjust its support based on the user's force and movement, providing just enough assistance to help the patient complete the movement without taking over completely. This allows the user (e.g., the user 106A) to attain the safety and control associated with the HMI device 104. The HMI device 104 may include, but not limited to social HMI devices, collaborative HMI device, prosthetics and exoskeletons, teleoperated HMI devices, and the like. Control may pass to end.

Although the flowchart 600 is illustrated as discrete operations, such as 604, 606, 608, 610, 612, 614, and 616 the disclosure is not so limited. Accordingly, in certain embodiments, such discrete operations may be further divided into additional operations, combined into fewer operations, or eliminated, depending on the implementation without detracting from the essence of the disclosed embodiments.

Various embodiments of the disclosure may provide a non-transitory computer-readable medium and/or storage medium having stored thereon, computer-executable instructions executable by a machine and/or a computer to operate an electronic device (e.g., the electronic device 102 of FIG. 1). Such instructions may cause the electronic device 102 to perform operations that may include receiving touch parameters (e.g., the touch parameters 210) associated with a physical-interaction of the HMI device 104 and a user (e.g., the user 106A). The operations may further include reception of control parameters (e.g., the control parameters 212) associated with an operator (e.g., the operator 106B) of the HMI device 104 to control the physical-interaction of the HMI device 104. The operations may further include control of the physical interaction of the HMI device 104, based on the received touch parameters 210 and the received control parameters 212. The operations may further include determination of a physical response of the user 106A, based on the control of the physical interaction of the HMI device 104, and determination of safety metrics associated with the user 106A, based on the determined physical response and the received touch parameters 204B. Also, the operations may further include determination of correlation information, based on the control of the physical-interaction of the HMI device and the determined safety metrics, and may further include control of the physical interaction of the HMI device 104, further based on the determined correlation information.

Exemplary aspects of the disclosure may provide an electronic device (such as the electronic device 102 of FIG. 1) that includes circuitry (such as the circuitry 202). The circuitry 202 may be configured to receive the touch parameters 210 associated with the physical-interaction of the HMI device 104 and the user 106A. Further, the circuitry 202 may be configured to receive control parameters 212 associated with the operator 106B of the HMI device 104 to control the physical-interaction of the HMI device 104 based on the received touch parameters 210 and the received control parameters 212. Further, the circuitry 202 may be configured to determine a physical response of the user 106A, based on the control of the physical interaction of the HMI device 104, and further determine safety metrics associated with the user 106A, based on the determined physical response and the received touch parameters 204B. Also, the circuitry 202 may be configured to determine correlation information based on the control of the physical-interaction of the HMI device and the determined safety metrics, and to control the physical-interaction of the HMI device and the user, further based on the determined correlation information.

The present disclosure may be realized in hardware, or a combination of hardware and software. The present disclosure may be realized in a centralized fashion, in at least one computer system, or in a distributed fashion, where different elements may be spread across several interconnected computer systems. A computer system or other apparatus adapted for carrying out the methods described herein may be suited. A combination of hardware and software may be a general-purpose computer system with a computer program that, when loaded and executed, may control the computer system such that it carries out the methods described herein. The present disclosure may be realized in hardware that includes a portion of an integrated circuit that also performs other functions. It may be understood that, depending on the embodiment, some of the steps described above may be eliminated, while other additional steps may be added, and the sequence of steps may be changed.

The present disclosure may also be embedded in a computer program product, which includes all the features that enable the implementation of the methods described herein, and which when loaded in a computer system is able to carry out these methods. Computer program, in the present context, means any expression, in any language, code or notation, of a set of instructions intended to cause a system with an information processing capability to perform a particular function either directly, or after either or both of the following: a) conversion to another language, code or notation; b) reproduction in a different material form. While the present disclosure has been described with reference to certain embodiments, it will be understood by those skilled in the art that various changes may be made, and equivalents may be substituted without departing from the scope of the present disclosure. In addition, many modifications may be made to adapt a particular situation or material to the teachings of the present disclosure without departing from its scope. Therefore, it is intended that the present disclosure is not limited to the particular embodiment disclosed, but that the present disclosure will include all embodiments that fall within the scope of the appended claims.

What is claimed is:

1. An electronic device, comprising:
circuitry configured to:
receive touch parameters associated with a physical-interaction of the HMI device and a user;
receive control parameters associated with an operator of the HMI device to control the physical-interaction of the HMI device;
control the physical interaction of the HMI device, based on the received touch parameters and the received control parameters;
determine a physical response of the user, based on the control of the physical interaction of the HMI device;
determine safety metrics associated with the user, based on the determined physical response and the received touch parameters;
determine correlation information based on the control of the physical-interaction of the HMI device and the determined safety metrics; and
control the physical-interaction of the HMI device and the user, further based on the determined correlation information.

2. The electronic device according to claim 1, wherein the touch parameters include at least one of:
a contact position of the HMI device on a body portion of the user,
a speed of the HMI device;
a force of the HMI device;
a softness of the HMI device; or
an orientation of the HMI device.

3. The electronic device according to claim 2, wherein the at least one of the safety metrics or a comfort metrics is negatively correlated with at least one of:
the speed of the HMI device,
the force of the HMI device, or
a level of safety associated with the contact position of the HMI device.

4. The electronic device according to claim 2, wherein the at least one of the safety metrics or a comfort metrics is positively correlated with at least one of:
the softness of the HMI device, or
the orientation of the HMI device.

5. The electronic device according to claim 1, wherein the control parameters include at least one of:
a user-control freedom parameter of the HMI device for the operator, or
a transparency factor of the HMI device for the operator.

6. The electronic device according to claim 1, wherein circuitry is further configured to:
determine a physiological stress of the user based on the determined safety metrics, wherein the determined physiological stress corresponds to the determined physical response of the user, and the physiological stress includes at least one of:
a gaze of the user,
a facial expression of the user, or
an involuntary motion of the user.

7. The electronic device according to claim 1, wherein the safety metrics is negatively correlated with risk metrics and positively correlated with a trust factor.

8. The electronic device according to claim 1, wherein the circuitry is further configured to:
receive background parameters of the user, wherein the background parameters include at least one of: a gender of the user, an age of the user, a purpose of touch associated with the HMI device, and a physical state of the user.

9. The electronic device according to claim 8, wherein the background parameters are correlated with at least one of the safety metrics or a comfort metrics.

10. The electronic device according to claim 8, wherein the circuitry is further configured to determine a task performance metrics associated with the HMI device, based on the received touch parameters and the received background parameters.

11. The electronic device according to claim 10, wherein the determined task performance metrics is positively correlated with a trust factor.

12. The electronic device according to claim 10, wherein the determined task performance metrics is positively correlated with the touch parameters.

13. The electronic device according to claim 1, wherein the control parameters associated with the operator have a positive correlation with at least one of a mental load associated with the user or a predictability of the HMI device.

14. A method, comprising:
in an electronic device:
receiving touch parameters associated with a physical-interaction of the HMI device and a user;
receiving control parameters associated with an operator of the HMI device to control the physical-interaction of the HMI device;
controlling the physical interaction of the HMI device, based on the received touch parameters and the received control parameters;
determining a physical response of the user, based on the control of the physical interaction of the HMI device;
determining safety metrics associated with the user, based on the determined physical response and the received touch parameters;
determining correlation information based on the control of the physical-interaction of the HMI device and the determined safety metrics; and
controlling the physical-interaction of the HMI device and the user, further based on the determined correlation information.

15. The method according to claim 14, wherein the touch parameters include at least one of:
a contact position of the HMI device on a body portion of the user,
a speed of the HMI device;
a force of the HMI device;
a softness of the HMI device; or
an orientation of the HMI device.

16. The method according to claim 15, wherein the at least one of the safety metrics or a comfort metrics is negatively correlated with at least one of:
the speed of the HMI device,
the force of the HMI device, or
a level of safety associated with the contact position of the HMI device.

17. The method according to claim 15, wherein the at least one of the safety metrics or a comfort metrics is positively correlated with at least one of:
the softness of the HMI device, or
the orientation of the HMI device.

18. The method according to claim 14, wherein the control parameters include at least one of:
a user-control freedom of the HMI device for the operator, or
a transparency factor of with the HMI device for the operator.

19. The method according to claim 14, further comprising:
determining a physiological stress of the user based on the determined safety metrics, wherein the determined physiological stress corresponds to the determined physical response of the user, and the physiological stress includes at least one of:
a gaze of the user,
a facial expression of the user, or
an involuntary motion of the user.

20. A non-transitory computer-readable medium having stored thereon, computer-executable instructions that when executed by an electronic device, causes the electronic device to execute operations, the operations comprising:
receiving touch parameters associated with a physical-interaction of the HMI device and a user;
receiving control parameters associated with an operator of the HMI device to control the physical-interaction of the HMI device;
controlling the physical interaction of the HMI device, based on the received touch parameters and the received control parameters;
determining a physical response of the user, based on the control of the physical interaction of the HMI device;
determining safety metrics associated with the user, based on the determined physical response and the received touch parameters;
determining correlation information based on the control of the physical-interaction of the HMI device and the determined safety metrics; and
controlling the physical-interaction of the HMI device and the user, further based on the determined correlation information.
